(19) United States
(12) Patent Application Publication
(10) Pub. No.: US 2017/0231594 A1
(43) Pub. Date: Aug. 17, 2017

(54) MEDICAL EXAMINATION SYSTEM

(71) Applicants: Lutz Dominick, Eggolsheim (DE); Vladyslav Ukis, Nürnberg (DE)

(72) Inventors: Lutz Dominick, Eggolsheim (DE); Vladyslav Ukis, Nürnberg (DE)

(21) Appl. No.: 15/433,140

(22) Filed: Feb. 15, 2017

(30) Foreign Application Priority Data
Feb. 16, 2016 (DE) 102016202335.7

(51) Int. Cl. A61B 6/00; A61B 5/055; A61B 8/00

(52) U.S. Cl. CPC A61B 6/5294; A61B 8/46; A61B 6/46; A61B 5/055

(57) ABSTRACT

A medical examination system for an imaging examination is provided. The medical examination system includes an imaging device for performing the imaging examination based on a scan protocol. The imaging device includes an interface for sending examination parameters and an interface for receiving scan protocols. The medical examination system further includes a central data store for storing a plurality of scan protocols. The central data store includes an interface for receiving the examination parameters from the imaging device, a selection entity for selecting a scan protocol from the plurality of stored scan protocols based on the received examination parameters, and an interface for sending the selected scan protocol to the imaging device.

### CROSS-REFERENCE TO RELATED APPLICATIONS

This application claims the benefit of DE 10 2016 202 335.7, filed on Feb. 16, 2016, which is hereby incorporated by reference in its entirety.

### TECHNICAL FIELD

Embodiments relate to a medical examination system for an imaging examination, an imaging device, a central data store, and a method for performing an imaging examination.

### BACKGROUND

In medical imaging devices such as, for example, magnetic resonance tomographs, computed tomographs, or ultrasound devices, a technical scan protocol for performing the examination is set manually at the respective device. The scan protocol is set at the device shortly before the imaging examination. For routine examinations, the corresponding scan protocol may be found by a search mechanism at the device. However, manual configuration of existing scan protocols is resource intensive and susceptible to error.

Search mechanisms, by which individual physical protocol parameters may be entered when searching for the scan protocol (e.g., a slice thickness or the number of slices to be recorded), exist. The equipment utilization plans may be supported by enterprise resource planning (ERP) systems. However, the ERP systems do not support scan protocols or allow selection of these scan protocols.

### SUMMARY AND DESCRIPTION

The scope of the present invention is defined solely by the appended claims and is not affected to any degree by the statements within this summary

The present embodiments may obviate one or more of the drawbacks or limitations in the related art. For example, the selection of a scan protocol for an imaging examination at a medical imaging device is simplified, such that a better image quality is achieved during the examination.

In an embodiment, a medical examination system is provided for an imaging examination. The medical examination system includes an imaging device for performing the imaging examination based on a scan protocol. The imaging device includes an interface for sending examination parameters and an interface for receiving scan protocols. The medical examination system also includes a central data store for storing a plurality of scan protocols. The central data store includes an interface for receiving the examination parameters from the imaging device, a selection entity for selecting a scan protocol from the plurality of stored scan protocols based on the received examination parameters, and an interface for sending the selected scan protocol to the imaging device. The medical examination system provides that manual setting of the scan protocol may be dispensed with and a high image quality is achieved based on the examination parameters for a plurality of devices.

In an embodiment, the imaging device is configured to transfer an examination image together with the scan protocol to the central data store. The resulting examination images may be used to improve the image quality further.

In an embodiment, the central data store is configured to analyze a quality of the examination image. Improved scan protocols may be identified automatically.

In an embodiment, the examination system is configured to remove personal patient information from the examination image. Personal data remains protected during the analysis of the examination images.

In an embodiment, the selection entity is configured to select the scan protocol based on a previous imaging examination of the patient. A suitable scan protocol may easily be found.

In an embodiment, the central data store is configured to determine an imaging device on which the selected scan protocol may be executed. The examination may be performed on a suitable device.

In an embodiment, the examination parameters include parameters relating to a patient age, a gender, a weight, a region of an organ or body, a previous diagnosis, an available imaging device with assigned protocols, a hospital-specific use of the imaging device, a utilization plan of the imaging device, and/or parameters for matching patient data against shared protocol databases within and outside the hospital chain. Suitable parameters may be taken into consideration for the selection of the scan protocol.

In an embodiment, the imaging device is a magnetic resonance tomography device, a computed tomography device, or an ultrasound device. Suitable devices are used to perform the medical examination based on the scan protocol.

In an embodiment, an imaging device is provided for performing the imaging examination based on a scan protocol. The imaging device includes an interface for sending examination parameters and an interface for receiving the scan protocol. This achieves the same advantages as the medical examination system according to one or more of the present embodiments.

In an embodiment, a central data store is provided for storing a multiplicity of scan protocols. The central data store includes an interface for receiving the examination parameters from an imaging device, a selection entity for selecting a scan protocol from the plurality of stored scan protocols based on the received examination parameters, and an interface for sending the selected scan protocol to an imaging device. This achieves the same advantages as the medical examination system according to one or more of the present embodiments.

In an embodiment, a method for performing an imaging examination is provided. The method includes sending examination parameters from an imaging device to a central data store, selecting a scan protocol from a plurality of stored scan protocols based on the received examination parameters in the central data store, and sending the selected scan protocol from the central data store to the imaging device. The manual setting of the scan protocol may be dispensed with, and a high image quality is achieved based on the examination parameters for a plurality of devices.

In an embodiment, the imaging device transfers an examination image together with the scan protocol to the central data store. The resulting examination images may be used to improve the image quality further.

In an embodiment, the central data store analyzes a quality of the examination image. Improved scan protocols may be identified automatically.

In an embodiment, the examination system removes personal patient information from the examination image. Personal data remains protected during the analysis of the examination images.

In an embodiment, the scan protocol is selected based on a previous imaging examination of the patient. A suitable scan protocol may be found.

### BRIEF DESCRIPTION OF THE DRAWINGS

FIG. 1 depicts a schematic diagram of a medical examination system according to an embodiment.

FIG. 2 depicts a further diagram of the medical examination system according to an embodiment.

FIG. 3 depicts a block diagram of a method for performing an imaging examination according to an embodiment.

### DETAILED DESCRIPTION

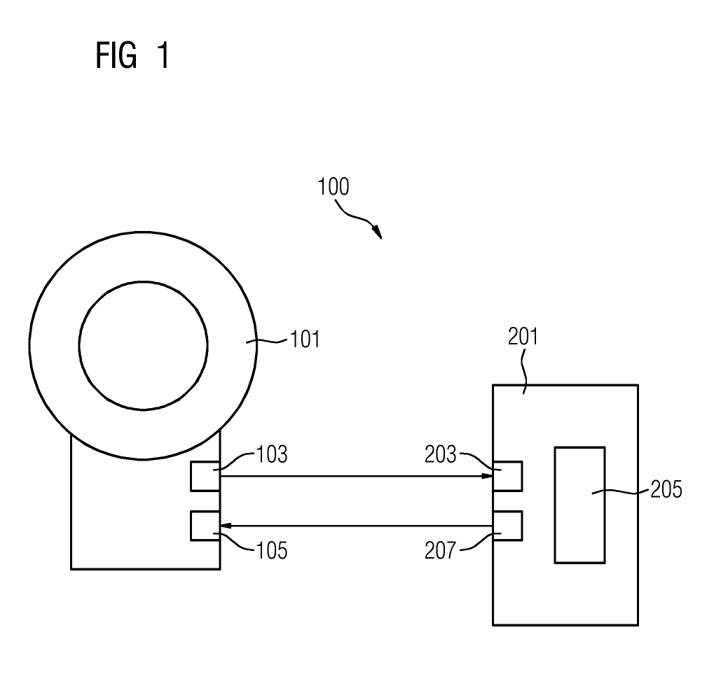

FIG. 1 depicts a schematic diagram of a medical examination system 100. The medical examination system 100 for an imaging examination includes an imaging device 101 for performing the imaging examination based on a scan protocol (e.g., a magnetic resonance tomography scanner (MRT scanner), a computed tomography scanner (CT scanner), or an ultrasound device).

The imaging device 101 performs the imaging examination based on the scan protocol, in which the technical parameters used by the device 101 during the examination are specified. The technical parameters of the scan protocol include details relating to technical device settings such as, for example, pulse duration and timed sequences of magnetic pulses (e.g., pulse sequences), gradient fields, or gantry angles. Configurable technical parameters to be used for the imaging examination on the imaging device 101 may be defined in the scan protocol.

An interface 103 for sending examination parameters and other data is provided at the imaging device 101. The examination parameters may be input manually at the imaging device 101 or extracted from an electronic medical file of the patient. The examination parameters and other data are sent to a central data store 201 (e.g., a cloud) via the interface 103. The data transmission is effected via a data line that is provided over the Internet, for example.

The examination parameters may include, for example, a patient age, a gender, a weight, a region of an organ or body (e.g., heart, thorax, abdomen, knee), a previous diagnosis, an available imaging device with the assigned protocols, a hospital-specific use of the imaging device, a utilization plan of the imaging device, and/or parameters for matching the patient data against shared protocol databases within and outside the hospital chain. The examination parameters may include other parameters.

A plurality of scan protocols for different examination parameters are stored in the central data store 201. The data store 201 includes a further interface 203 for receiving the examination parameters from the imaging device 101. The central data store 201 may be connected to a plurality of imaging devices 101, for which the data store 201 provides corresponding scan protocols.

In a selection entity 205, a scan protocol is selected from the plurality of scan protocols based on the received examination parameters. The automatic selection entity 205 takes the form of a hardware or software component, for example, and uses a configurable set of examination parameters relating to the medical case as input parameters in order to select a suitable scan protocol for the imaging device 101. With the examination parameters from different dimensions, the scan protocols from the central data store 201 may be used in a shared manner based on a cloud or in-house.

The central data store 201 further includes an interface 207 for sending the selected scan protocol to the imaging device 101. The imaging device 101 receives the scan protocol sent via a corresponding interface 105 and performs the imaging examination using the received scan protocol.

By virtue of the shared use of a tried and tested scan protocol, a predictable and repeatable scan duration and image quality are achieved. By virtue of the central data store 201, the scan protocols for all imaging devices 101 may be separated from the imaging devices 101, and the scan protocols may be retrieved from any location by the imaging device 101. The scan protocols may be stored in the central data store 201 together with the respective optimal image results and additional acts in order to perform further automation.

The medical examination system 100 allows the scan protocol to be set in a fully automatic manner, irrespective of supply point, and allows an optimal setting with high image quality. In addition, an option may be provided for improving the scan protocol again at a later time. The scan protocol may be checked again manually by a user before an examination.

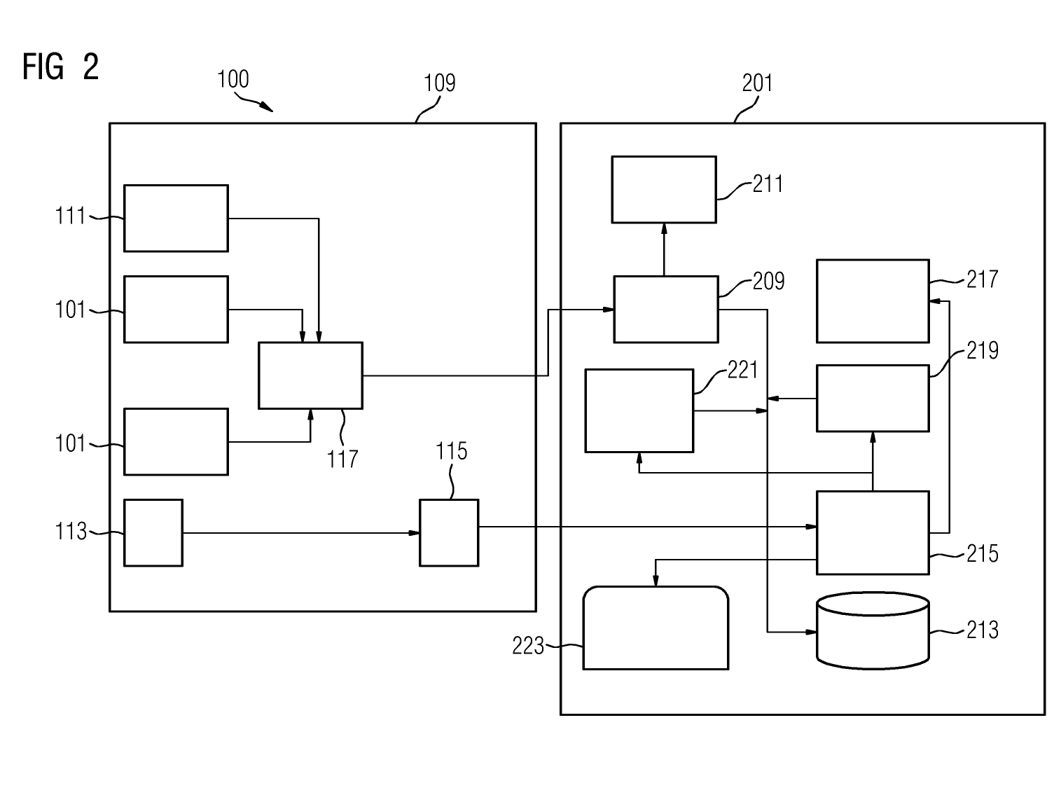

FIG. 2 depicts a further diagram of the medical examination system 100. The imaging devices 101, a picture archiving and communication system (PACS) 113, a hospital information system (HIS) 113, and a radiology information system (RIS) 115 are arranged in a hospital 109.

The scan protocol may be transferred from the imaging devices 101 or the picture archiving and communication system 113 to an upload component 117 for the scan protocol and data in the context of a study. The data that is transferred from the imaging devices 101 or the picture archiving and communication system 111 to an upload component 117 may include, for example, a DICOM study, a series, the recorded examination images, and the method acts performed by the imaging device 101 (Modality Performed Procedure Step: MPPS).

Within the upload component 117, a series may be processed and calculated to extract protocol information or to process example studies. The upload component 117 anonymizes the data the upload component 117 receives, and transfers the data to the central data store 201 (e.g., a cloud). The data may include a number of examination images in combination with a quality level that is assigned to the respective examination image.

The upload component 117 for scan protocols and data sends patient information, study information, scan protocols, image quality data and device data to the central data store 201 in a minimized and anonymized manner, allowing configuration for other medical cases subsequently. In an embodiment, data mining may be used to obtain data from the picture archiving and communication system 113, and generate the background information from an existing DICOM hierarchy and MPPS, for example.

The central data store 201 includes a receive component 209 for the scan protocol and the data. The received data is stored in a storage component 211. Index data is transferred to an index database 213, in which the device model, examination images, protocols, and the assigned quality level are stored.

An examination (scan) of a patient is planned in the hospital information system 113. The name, the gender, a date of birth and a patient identification number as a quadruple, or further examination parameters are recorded.

Suitable scan protocols are obtained from a protocol retrieval component 215 in the central data store 201. The protocol retrieval component 215 is based on a protocol search algorithm, and processes a configurable and flexible set of dimensions as examination parameters. The examination parameters are cross-referenced to each other by the algorithm to perform the task of protocol selection automatically.

One possible set of dimensions includes, for example, patient information including age, weight and known protocol-related parameters, a diagnosis (e.g., a previous diagnosis) with symptoms and organ information, previous diseases (e.g., priors), device data and scan protocols, a personal patient scan protocol profile, departmental guidelines relating to optimal protocol use, examination images having a verified quality and the original protocol information, an Internet search engine search for protocol optimization, and a crowd-based optimal use of scan protocols and an image quality.

The protocol search algorithm works with a number of dedicated medical dimensions to find a scan protocol. The dimensions may be thought of as petals of a daisy that have the scan protocol as a parameter. The petals hold a subset of the patient-related parameters cited above, while the center represents the actual scan protocol. There are a number of petals that point to a scan protocol if the volume of data in the dimensions is large enough.

The dimensions initially record domain objectives such as high image quality, for example. The dimensions are combined with protocol information and coordinated with a number of medical cases, allowing the dimensions to be configured for a new patient case and find protocol information. The use of a plurality of dimensions makes the protocol search algorithm reliable and records all of the important medical considerations for selecting the scan protocol.

Examples of crowd based optimal use of scan protocols and image quality are crowdsourcing (e.g. outsourcing the task to a group) based on scan evaluations by the whole group, crowdsourcing based on scan evaluations of radiologists by the whole group, crowdsourcing based on scan evaluations according to geographical location, crowdsourcing based on scan evaluations of radiologists according to geographical location, crowdsourcing based on the number of supporters of a radiologist, or crowdsourcing based on the number of shares received by a study.

Embodiments may be compatible with processing scan results in data stores outside of the location of the imaging device 101, including a DICOM RDSR standard, or using portable scan protocol formats that may allow manufacturer independent use.

The automated selection of the scan protocol is performed by the protocol retrieval component 215 and is based primarily on patient data and the details of the medical case (e.g., an illness, a previous diagnosis, or an organ) and not primarily on the imaging device 101. In one embodiment, the output of the protocol retrieval component 215 is a list of protocol names offering technical compatibility with an existing DICOM/HL7-based data flow in hospitals. In a further embodiment, access to complete scan protocols is enabled.

One input into the protocol retrieval component 215 is the patient information and the diagnosis (e.g., previous diagnosis) including organ region and body region, based on standardized medical codes. The protocol retrieval component 215 matches the information against all other dimensions with the aid of further components and sources, and proposes a list of scan protocols for the available devices in the hospital.

The output calculation may be configured. One example is the most frequent occurrence statistically of one or more scan protocols for the specified patient and the medical case, based on the protocol lists that have been output for the other components involved. The output calculation may be configured in order to use dedicated optimal scan protocols that are known in the facility, allowing for an optimal method to be used automatically.

Using the range of statistics for the patient and the diagnosis, a crowd based information store is searched to obtain further options for the scan protocol selection. The medical objective is to allow the best treatment based on the best examination image.

The patient may be assigned to one of the proposed devices 101 automatically. The final medical decision with respect to the scan protocol may ultimately be made at the time of examination according to the condition of the patient. The examination results (e.g., scan results) are then likewise sent to the central data store 201. The protocol retrieval component 215 may likewise propose devices and protocols for further treatment.

A patient identification component 217 matches the current patient against a demographic set of similar patients with the same diagnosis to extend the range of statistics (e.g., a patient age group that may be searched for the optimal scan protocols). The input into the patient identification component 217 is configurable patient information (e.g., gender, age, diagnosis or organ), and the output is a range of statistically similar ages, a list of protocols, and body regions. The protocol retrieval component 215 receives a patient range to be searched by using the patient identification component 217.

A previous study search component 219 includes previous studies of the current patient to gather a personal protocol for the patient and, for example, to determine a priority for reuse of the same scan protocol if a series of examinations is to be provided. The previous study search component 219 may be used to calculate a cumulative patient dosage as an input for scan protocol selection and planning. The input may be retrieved directly from the picture archiving and communication system 113 (e.g., gender, age, weight, diagnosis, organ). A personal list of scan protocols and body regions is output. The protocol retrieval component 215 receives previous studies relating to the patient using the previous study search component 219.

A quality image search component 221 matches any chosen scan protocols and device information with examination images of high quality. A configurable list of medical case information (e.g., region or organ) is input. Protocol information and image examples are output. Using the quality image search component 221, the protocol retrieval component 215 receives similar examination images.

A crowdbased protocol store 223 supplements the quality image search component 221. The crowd-based protocol store 223 includes in-house and publicly available optimal examination images and the associated protocol information. The input is a configurable list of medical case information (e.g., region or organ). Protocol information and image examples are output. Using the crowd-based protocol store 223, the protocol retrieval component 215 receives suitable scan protocols.

The automated selection mechanism includes a protocol search algorithm with a number of software components. The selection mechanism uses a configurable set of input parameters from the medical case, and matches the the set of input parameters with a configurable set of medical dimensions. Since the scan protocols and the imaging devices 101 are separate, the recording workstation may retrieve optimal scan protocols from an external source. The automated preselection of scan protocols is already made before the recording workstation is reached in the clinical workflow. The preselection may also be performed before the patient is examined at the recording workstation.

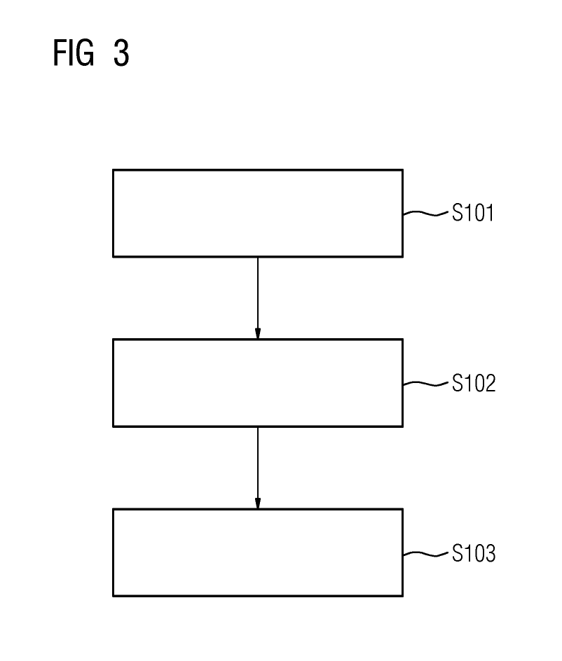

FIG. 3 depicts a block diagram of a method for performing an imaging examination. The method includes act S101 where examination parameters are sent from the imaging device 101 to the central data store 201. In act S102, the scan protocol is selected from a plurality of stored scan protocols by the selection entity 205 based on the received examination parameters in the central data store 201. In act S103, the selected scan protocol is sent from the central data store 201 to the imaging device 101.

By virtue of the medical examination system 100, manual protocol selection is automated, avoiding manual configuration of the scan protocol. The selection of the supply point is automated based on optimal methods.

An early configuration of an examination to optimal scan protocols allows for predicting how the imaging device 101 may be used in order to obtain optimal examination images. In addition to the examination images of high quality and the optimization of a medical treatment, the early automatic configuration of the examination and the protocol represents a possible input into the planning software of the hospital, that is used for the optimized planning of operating procedures. An embodiment provides an automatic protocol proposal that is available for the purpose of treatment planning before a scan, during a scan, or after a scan, for example, and is compatible with a clinical workflow.

The method already defines a protocol range at RIS level, based on a patient quadruple or previous diagnosis data. The method may also use a suitable imaging device 101 using knowledge from and within the central data store 210 (e.g., the cloud). The examination (e.g., case) of the patient specifies the scan protocol and makes the scan protocol available to the imaging device 101, at which the information may be imported automatically and transformed for the device 101.

The scan protocol is completely separate from the imaging device 101 in technical terms and may be set together with the scan request. The scanning workstation at the imaging device 101 allows the export and import of new scan protocols. In effect, the full protocol information is appended to the series for all modalities, going beyond a DICOM, RDSR and MPPS definition.

The medical examination system 100 for the imaging examination represents an automated, autonomous, configurable, self-configuring, self-optimizing, cloud-based mechanism for selecting a scan protocol. The mechanism is independent of a supply point and may be used at any time in order to allow an optimal protocol selection and thereby provide a high image quality. Customer loyalty may be improved on the part of patients, and the reputation of the examining facility may be improved.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present invention has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A medical examination system for an imaging examination, the medical examination system comprising:
an imaging device configured to perform the imaging examination based on a scan protocol, the imaging device comprising:
a first interface, the first interface configured to send examination parameters; and
a second interface, the second interface configured to receive scan protocols;
a central data store configured to store a plurality of scan protocols, the central data store comprising:
a third interface, the third interface configured to receive the examination parameters from the imaging device;
a selection entity configured to select the scan protocol from the plurality of stored scan protocols based on the received examination parameters; and
a fourth interface, the fourth interface configured to send the selected scan protocol to the imaging device.

2. The medical examination system of claim 1, wherein the imaging device is further configured to transfer an examination image together with the scan protocol to the central data store.

3. The medical examination system of claim 2, wherein the central data store is further configured to analyze a quality of the examination image.

4. The medical examination system of claim 2, wherein the medical examination system is configured to remove personal patient information from the examination image.

5. The medical examination system of claim 1, wherein the selection entity is further configured to select the scan protocol based on a previous imaging examination of the patient.

6. The medical examination system of claim 1, wherein the central data store is further configured to determine a type of imaging device on which the selected scan protocol is executable.

7. The medical examination system of claim 1, wherein the examination parameters comprise parameters relating to a patient age, a gender, a weight, a region of an organ or body, a previous diagnosis, an available imaging device with assigned protocols, a hospital-specific use of the imaging device, a utilization plan of the imaging device, parameters for matching patient data against shared protocol databases within and outside a hospital chain, or any combination thereof.

8. The medical examination system of claim 1, wherein the imaging device is a magnetic resonance tomography device, a computed tomography device, or an ultrasound device.

9. An imaging device for performing an imaging examination based on a scan protocol, the imaging device comprising:
a first interface configured to send examination parameters; and
a second interface configured to receive the scan protocol.

10. A central data store for storing a plurality of scan protocols, the central data store comprising:
a first interface configured to receive examination parameters from an imaging device;
a selection entity configured to select a scan protocol from the plurality of stored scan protocols based on the received examination parameters; and
a second interface configured to send the selected scan protocol to the imaging device.

11. A method for performing an imaging examination, the method comprising:
sending examination parameters from an imaging device to a central data store;
selecting a scan protocol from a plurality of stored scan protocols based on the examination parameters in the central data store; and
sending the selected scan protocol from the central data store to the imaging device.

12. The method of claim 11, further comprising:
transfering, by the imaging device, an examination image together with the selected scan protocol to the central data store.

13. The method of claim 12, further comprising:
analyzing, by the central data store, a quality of the examination image.

14. The method of claim 12, further comprising:
removing personal patient information from the examination image.

15. The method of claim 11, wherein the scan protocol is selected further based on a previous imaging examination of a patient.

16. The method of claim 11, further comprising:
determining a type of imaging device to execute the selected scan protocol.

17. The method of claim 11, wherein the examination parameters comprise parameters relating to a patient age, a gender, a weight, a region of an organ or body, a previous diagnosis, an available imaging device with assigned protocols, a hospital-specific use of the imaging device, a utilization plan of the imaging device, parameters for matching patient data against shared protocol databases within and outside a hospital chain, or any combination thereof.

18. The method of claim 11, wherein the imaging device is a magnetic resonance tomography device, a computed tomography device or an ultrasound device.
